(19) United States
(12) Patent Application Publication
(10) Pub. No.: US 2018/0273464 A1
(43) Pub. Date: Sep. 27, 2018

(54) SYNTHESIS AND IN VITRO ACTIVITY OF D-LACTIC ACID OLIGOMERS

(71) Applicants: Joel Steven Goldberg, Hillsborough, NC (US); David Martin Gooden, Hillsborough, NC (US)

(72) Inventors: Joel Steven Goldberg, Hillsborough, NC (US); David Martin Gooden, Hillsborough, NC (US)

(21) Appl. No.: 15/990,011

(22) Filed: May 25, 2018

(51) Int. Cl. C07C 67/465; C07C 69/68

(52) U.S. Cl. CPC C07C 67/465; C07C 69/68

(57) ABSTRACT

This invention describes the synthesis and pharmacologic activity of n=2, n=3, n=4, n=5, and n=6 D-lactic acid oligomers. D-lactic acid dimer has pharmacologic activity and sequesters L-lactate, and the other D-lactic acid oligomers have no activity in vitro, but may have pharmacologic activity in vivo as prodrugs of D-lactic acid dimer.

### CROSS-REFERENCES TO RELATED APPLICATIONS

None

### FEDERALLY FUNDED RESEARCH

None

### BACKGROUND OF THE INVENTION

In 2011, at Duke University and the Durham Veterans Affairs Medical Center, a stereocomplex chemical reaction was discovered between L-lactate and Polymer D-lactic acid (PDLA). (Goldberg, Weinberg 2014, 2016) A stereocomplex is a new substance that is formed when a molecule and its mirror image (D and L or enantiomers) come together with the proper orientation. The reaction occurs spontaneously, and rapidly without a catalyst or enzyme. Later it was discovered that D-lactic acid dimer (two D-lactic acids connected by an ester bond) would “sequester or trap” L-lactate. (Goldberg, Weinberg 2017)

L-lactate has many potential functions. It can act as a neurotransmitter, a buffer for the efflux of hydrogen ions in some cancer cells, and an intermediary in glycolysis. D-lactic acid dimer in a mixture of D-lactic acid oligomers was shown to have analgesic properties when applied to an open wound. (Goldberg 2014, 2015a) As a hydrogel, D-lactic acid dimer could be applied to nerves, sequestering lactate, thereby providing extended analgesia. The nerves most likely to be affected would be nociceptors that depend on L-lactate for energy to repetitively fire. Thus, D-lactic acid dimer may be selective for nociceptors and provide analgesia for many pain conditions in which a peripheral nerve or nerves may be a generator of pain.

D-lactic acid dimer in a mixture of D-lactic acid oligomers and as a single agent was shown to kill cultured leukemia, HeLa, and retinoblastoma cells. (Goldberg, Weinberg 2016, 2017) D-lactic acid dimer was less cytotoxic than L-lactic at a similar hydrogen ion concentration when applied to normal fibroblasts. The cytotoxic effects of D-lactic acid dimer were believed to be from sequestration of L-lactic acid and selective to cancer cells via the Warburg effect. (Goldberg, Weinberg 2017) Since L-lactate is an intermediary in glycolysis, it is anticipated that D-lactic acid dimer would disrupt hypermetabolic systems such as occur in malarial infections that utilize L-lactate as a fuel or hydrogen ion efflux. (Goldberg 2015b)

We believe that D-lactic acid dimer would be unlikely to produce effects as a systemically administered drug, because the concentration required to form the stereocomplex may not be realistically obtainable by the oral or parenteral route. However, local application of D-lactic acid dimer onto or into tumors could be tumoricidal and useful as a debulking agent. Local application of chemotherapeutic drugs has been performed during neurosurgery where topical application of carmustine implants, Gliadel® Wafers treat glioblastomas. (Perry et al 2007) Also, cutaneous malignancies in which D-lactic acid dimer is directly applied to lesions may be a therapeutic option and replace topical 5 fluorouracil which has many side effects.

Because a more potent drug than D-lactic acid dimer could exist with greater than two D-lactic acid units, we decided to synthesize n=3, n=4, n=5, and n=6 D-lactic acid oligomers and test the D-lactic acid oligomers for pharmacologic activity.

### DESCRIPTION OF THE DRAWINGS

FIGS. 1-4 show the synthesis of partially protected D-lactic acid dimer from methyl D-(+)-lactate.

FIGS. 5-7 show the synthesis of D-lactic acid trimer from the partially protected D-lactic acid dimer.

FIGS. 8-10 show the synthesis of partially protected D-lactic acid tetramer from the protected D-lactic acid trimer.

FIGS. 10-13 show the synthesis of D-lactic acid pentamer from the partially protected D-lactic acid tetramer.

FIGS. 14-17 shows the synthesis of D-lactic acid hexamer from the protected D-lactic acid pentamer.

### DETAILED DESCRIPTION OF THE INVENTION

Synthesis of n=2, n=3, n=4, n=5, and n=6 D-Lactic Acid Oligomers

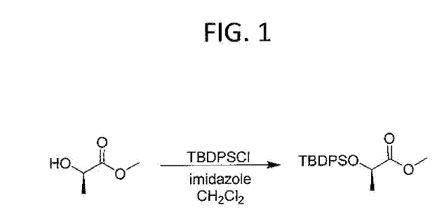

A solution of methyl D-(+)-lactate (25 g, 241 mmol) and imidazole (21 g, 313 mmol, 1.3 eq.) in CH₂Cl₂ (125 mL) was cooled in an ice-NaCl bath. Tert-butyldiphenylchlorosilane (97%, 66 mL, 253 mmol, 1.05 eq.) was added dropwise over 30 minutes. The resulting slurry was stirred for an additional 3 h after which time analysis by TLC (100% CH₂Cl₂, KMnO₄ staining) indicated the starting alcohol had been completely consumed. The reaction was diluted with CH₂Cl₂ (400 mL) and brine (500 mL). Following phase separation, the aqueous phase was extracted with CH₂Cl₂ (200 mL). The combined extracts were dried (MgSO₄). The drying agent was removed by filtration and the filtrate was concentrated to dryness under reduced pressure giving the silylether as a translucent, pale yellow oily-liquid (82 g, 99%). FIG. 1

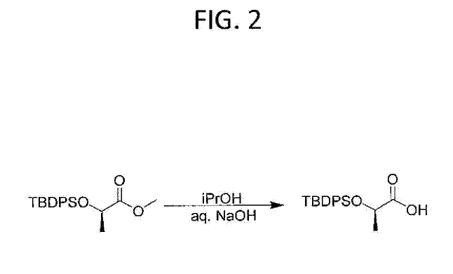

A solution of NaOH (19.3 g, 482 mmol, 2 eq.) in H₂O (250 mL) was added in one portion to a solution of the methyl ester (82 g, 240 mmol) in iPrOH (600 mL). The reaction mixture was stirred for 1.5 h after which time analysis by TLC (100% CH₂Cl₂) indicated essentially complete consumption of the starting ester. The mixture was poured into brine (1200 mL) and extracted with hexane (2×300 mL). This extraction was done to remove the slight excess of starting material as well as a few unidentified nonpolar impurities. The aqueous phase was acidified to pH˜2-3 (universal indicating pH paper) with 12M HCl, and extracted with hexane (2×500 mL). The combined extracts were washed with brine (2×400 mL) and dried (MgSO₄). The drying agent was removed by filtration and the filtrate was concentrated to dryness under reduced pressure giving the crude acid as a thick, clear pale oil [crystallized after standing 5-6 days at room temperature] (75 g, 95%) which was used in the next step without further purification. FIG. 2

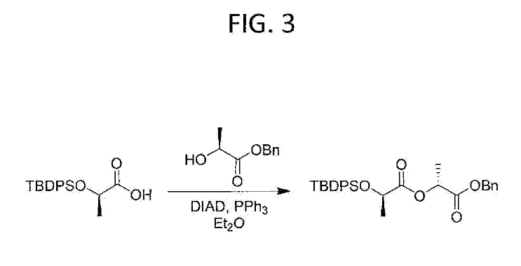

Diisopropyl azodicarboxylate (94%, 16 mL, 75 mmol, 1.5 eq.) was added dropwise over 20 minutes to an ice bath cooled solution of benzyl (S)-(−)-lactate (9.0 g, 50 mmol), the TBDPS protected (R)-lactic acid (16.4 g, 50 mmol) and PPh3 (19.7 g, 75 mmol, 1.5 eq.) in Et₂O (1 L). After 30 min, the cooling bath was removed. After an additional 30-60 minutes, analysis of the reaction by TLC (30% EtOAc in hexanes) indicated complete consumption of the benzyl (S)-(−)-lactate. Silica gel (˜30 g) was added and the reaction mixture was concentrated to dryness under reduced pressure. Flash column chromatography (RediSepR_{f} SiO₂ (220 g), 100% hexanes→5% EtOAc in hexanes, monitoring at 220 and 254 nm) gave the fully protected dimer as a transparent oil (16.2 g, 66%). FIG. 3

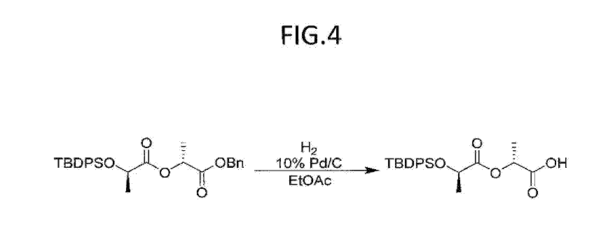

A suspension of the benzyl ester (16.2 g, 33 mmol) and 10% Pd/C (1.8 g) in EtOAc (100 mL) was stirred overnight under a balloon of H₂ after which time analysis of the reaction mixture by TLC (10% EtOAc in hexanes) indicated complete consumption of starting material. Celite (˜5 g) was added and, after 5 minutes of stirring, the mixture was filtered through a pad of Celite. The pad was washed with EtOAc (2×50 mL) and the filtrate was concentrated to dryness under reduced pressure giving the product as a transparent oil (13.2 g, 99%). FIG. 4

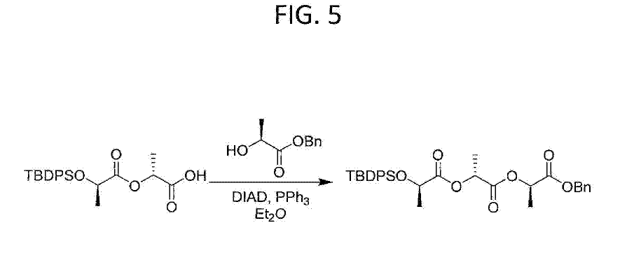

Diisopropyl azodicarboxylate (94%, 9.8 mL, 50 mmol, 1.5 eq.) was added dropwise over 10 minutes to an ice bath cooled solution of benzyl (S)-(−)-lactate (5.9 g, 33 mmol), the TBDPS protected (R)-lactic acid dimer (13.2 g, 33 mmol) and PPh3 (13.1 g, 50 mmol, 1.5 eq.) in Et₂O (650 mL). After 30 min, the cooling bath was removed. After an additional 30-60 minutes, analysis of the reaction by TLC (30% EtOAc in hexanes) indicated complete consumption of the benzyl (S)-(−)-lactate. Silica gel (˜20 g) was added and the reaction mixture was concentrated to dryness under reduced pressure. Flash column chromatography (RediSepR_{f} SiO₂ (220 g), 100% hexanes→5% EtOAc in hexanes over 30 min, monitoring at 220 and 254 nm) gave the fully protected trimer as a transparent oil (17.4 g, 94%). FIG. 5

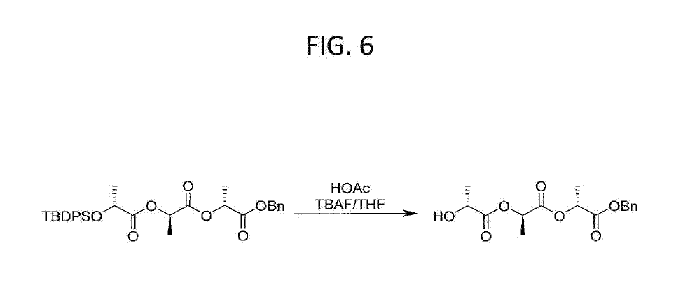

Glacial acetic acid (5.9 mL, 100 mmol, 12.7 eq.) was added to a solution of the silyl protected trimer (4.3 g, 7.6 mmol) in THF (35 mL). A solution of TBAF (1.0 M in THF, 17 mL, 17 mmol, 2.2 eq.) was added slowly and the reaction mixture was stirred at room temperature for approximately 2 h after which time, analysis by TLC (10% EtOAc in hexanes) indicated essentially complete consumption of starting material. The mixture was partitioned between ethyl acetate (150 mL) and brine (150 mL). The organic phase was washed with saturated aqueous NaHCO₃ (2×150 mL), aqueous 10% citric acid (150 mL), and brine (150 mL). The organic layer was drained onto silica gel (˜10 g) and the mixture was concentrated to dryness under reduced pressure. Flash column chromatography (RediSepR_{f} SiO₂ (40 g), 100% hexanes→40% EtOAc in hexanes, monitoring at 220 and 254 nm and visualizing fractions with KMnO₄ stain) gave the OH-trimer as a clear oil (2.2 g, 89%). FIG. 6

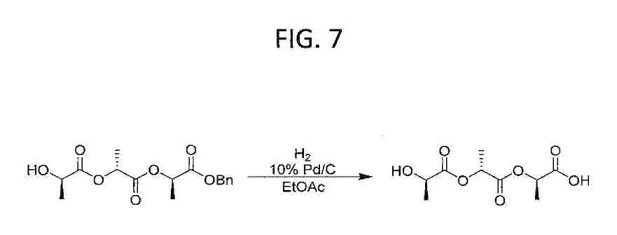

A suspension of the benzyl ester (2.0 g, 6.2 mmol) and 10% Pd/C (300 mg) in EtOAc (20 mL) was stirred overnight under a balloon of H₂ after which time analysis of the reaction mixture by TLC (30% EtOAc in hexanes) indicated complete consumption of starting material. The mixture was filtered through a pad of Celite. The pad was washed with EtOAc (2×10 mL) and the filtrate was concentrated to dryness under reduced pressure giving the trimer as a clear oil which crystallized while on the high vacuum line overnight (1.4 g, 97%). FIG. 7

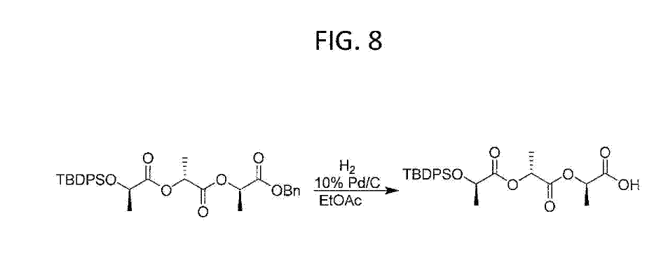

A suspension of the benzyl ester (17.3 g, 31 mmol) and 10% Pd/C (2 g) in EtOAc (100 mL) was stirred overnight under a balloon of H₂ after which time analysis of the reaction mixture by TLC (5% EtOAc in hexanes) indicated complete consumption of starting material. Celite (˜5 g) was added and, after 5 minutes of stirring, the mixture was filtered through a pad of Celite. The pad was washed with EtOAc (2×50 mL) and the filtrate was concentrated to dryness under reduced pressure giving the product as a transparent oil (14.7 g, 100%). FIG. 8

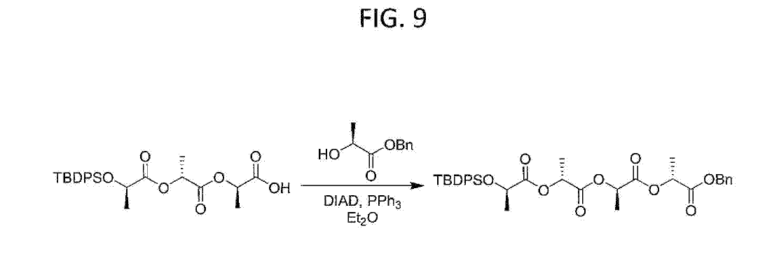

Diisopropyl azodicarboxylate (98%, 8.1 mL, 41 mmol, 1.5 eq.) was added dropwise over 10 minutes to an ice bath cooled solution of benzyl (S)-(−)-lactate (4.9 g, 27.1 mmol), the TBDPS protected (R)-lactic acid trimer (12.8 g, 27.1 mmol) and PPh3 (10.7 g, 41 mmol, 1.5 eq.) in Et₂O (500 mL). After 30 min, the cooling bath was removed. After an additional 30-60 minutes, analysis of the reaction by TLC (30% EtOAc in hexanes) indicated complete consumption of the benzyl (S)-(−)-lactate. Silica gel (˜20 g) was added and the reaction mixture was concentrated to dryness under reduced pressure. Flash column chromatography (RediSepR_{f} SiO₂ (220 g), 100% hexanes→10% EtOAc in hexanes over 30 min, monitoring at 220 and 254 nm) gave the fully protected tetramer as a transparent oil (15.6 g, 91%). FIG. 9

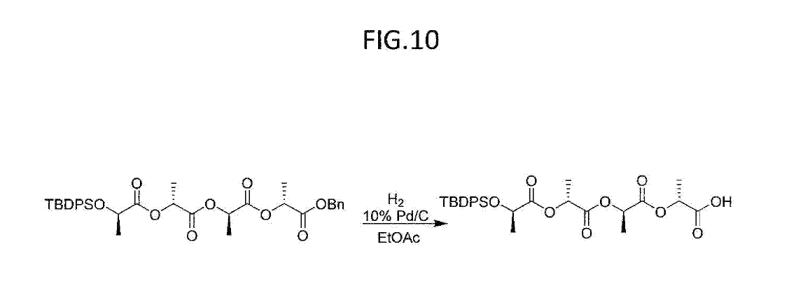

A suspension of the benzyl ester (15.6 g, 24.6 mmol) and 10% Pd/C (2 g) in EtOAc (100 mL) was stirred overnight under a balloon of H₂ after which time analysis of the reaction mixture by TLC (5% EtOAc in hexanes) indicated complete consumption of starting material. Celite (˜5 g) was added and, after 5 minutes of stirring, the mixture was filtered through a pad of Celite. The pad was washed with EtOAc (2×50 mL) and the filtrate was concentrated to dryness under reduced pressure giving the product as a transparent oil (13 g, 97%). FIG. 10

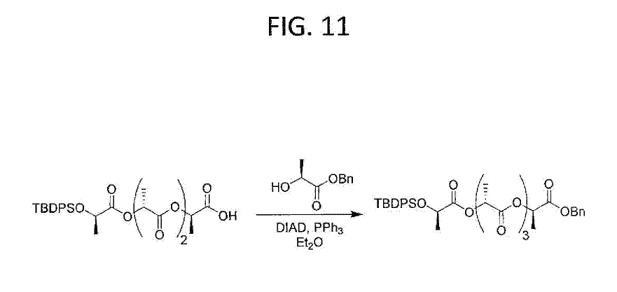

Diisopropyl azodicarboxylate (98%, 7.0 mL, 35.9 mmol, 1.5 eq.) was added dropwise over 10 minutes to an ice bath cooled solution of benzyl (S)-(−)-lactate (4.3 g, 23.9 mmol), the TBDPS protected (R)-lactic acid tetramer (13 g, 23.9 mmol) and PPh3 (9.4 g, 35.9 mmol, 1.5 eq.) in Et₂O (500 mL). After 30 min, the cooling bath was removed. After an additional 30-60 minutes, analysis of the reaction by TLC (30% EtOAc in hexanes) indicated complete consumption of the benzyl (S)-(−)-lactate. Silica gel (˜20 g) was added and the reaction mixture was concentrated to dryness under reduced pressure. Flash column chromatography (RediSepR_{f} SiO₂ (220 g), 100% hexanes→10% EtOAc in hexanes over 30 min, monitoring at 220 and 254 nm) gave the fully protected pentamer as a transparent oil (13.1 g, 78%). FIG. 11

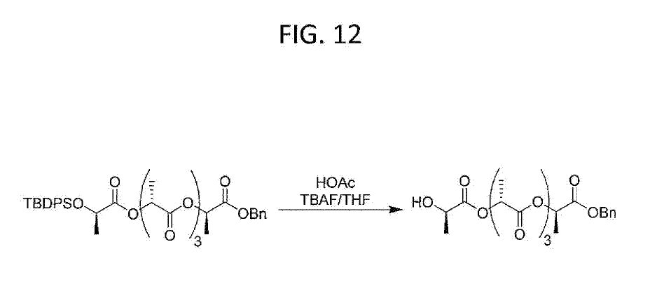

Glacial acetic acid (6.4 mL, 112 mmol, 12.7 eq.) was added to a solution of the silyl protected pentamer (6.2 g, 8.8 mmol) in THF (40 mL). A solution of TBAF (1.0 M in THF, 20 mL, 20 mmol, 2.2 eq.) was added slowly and the reaction mixture was stirred at room temperature for approximately 2 h after which time, analysis by TLC (10% EtOAc in hexanes) indicated essentially complete consumption of starting material. The mixture was partitioned between ethyl acetate (150 mL) and brine (150 mL). The organic phase was washed with saturated aqueous NaHCO₃ (2×150 mL), aqueous 10% citric acid (150 mL), and brine (150 mL). The organic layer was drained onto silica gel (˜10 g) and the mixture was concentrated to dryness under reduced pressure. Flash column chromatography (RediSepR_{f} SiO₂ (40 g), 100% hexanes→40% EtOAc in hexanes, monitoring at 220 and 254 nm and visualizing fractions with KMnO₄ stain) gave the OH-pentamer as a clear oil (3.6 g, 88%). FIG. 12

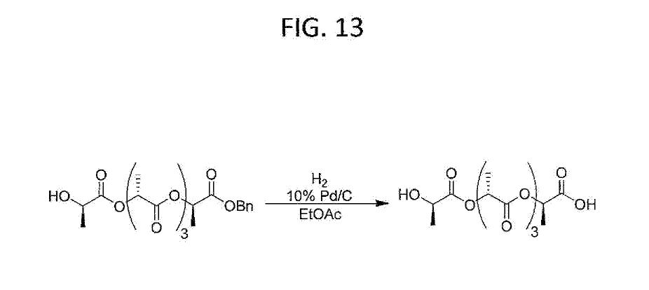

A suspension of the benzyl ester (3.5 g, 7.5 mmol) and 10% Pd/C (300 mg) in EtOAc (20 mL) was stirred overnight under a balloon of H₂ after which time analysis of the reaction mixture by TLC (30% EtOAc in hexanes) indicated complete consumption of starting material. The mixture was filtered through a pad of Celite. The pad was washed with EtOAc (2×10 mL) and the filtrate was concentrated to dryness under reduced pressure giving the pentamer as a clear pale oil (g, 95%). FIG. 13

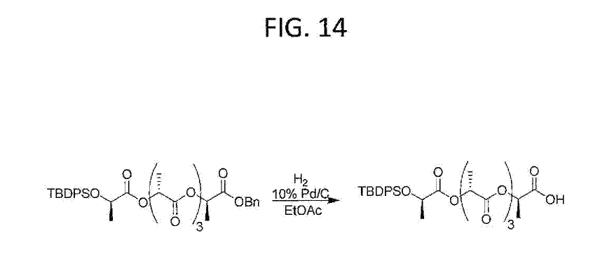

A suspension of the benzyl ester (6.4 g, 9 mmol) and 10% Pd/C (0.7 g) in EtOAc (30 mL) was stirred overnight under a balloon of H₂ after which time analysis of the reaction mixture by TLC (5% EtOAc in hexanes) indicated complete consumption of starting material. Celite (˜5 g) was added and, after 5 minutes of stirring, the mixture was filtered through a pad of Celite. The pad was washed with EtOAc (2×50 mL) and the filtrate was concentrated to dryness under reduced pressure giving the product as a transparent oil (5.5 g, 100%). FIG. 14

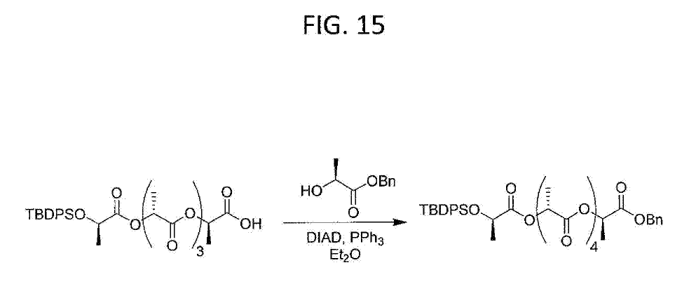

Diisopropyl azodicarboxylate (98%, 2.6 mL, 13.4 mmol, 1.5 eq.) was added dropwise over 10 minutes to an ice bath cooled solution of benzyl (S)-(−)-lactate (1.6 g, 8.9 mmol), the TBDPS protected (R)-lactic acid pentamer (5.5 g, 8.9 mmol) and PPh3 (3.5 g, 13.4 mmol, 1.5 eq.) in Et₂O (500 mL). After 30 min, the cooling bath was removed. After an additional 30-60 minutes, analysis of the reaction by TLC (30% EtOAc in hexanes) indicated complete consumption of the benzyl (S)-(−)-lactate. Silica gel (˜20 g) was added and the reaction mixture was concentrated to dryness under reduced pressure. Flash column chromatography (RediSepR_{f} SiO₂ (80 g), 100% hexanes→20% EtOAc in hexanes over 30 min, monitoring at 220 and 254 nm) gave the fully protected hexamer as a transparent oil (6.0 g, 86%). FIG. 15

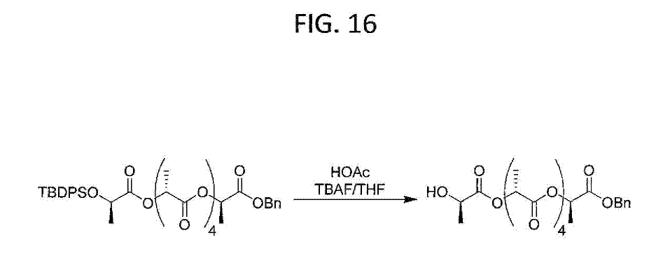

Glacial acetic acid (5.6 mL, 98 mmol, 12.7 eq.) was added to a solution of the silyl protected hexamer (6.0 g, 7.7 mmol) in THF (35 mL). A solution of TBAF (1.0 M in THF, 17 mL, 17 mmol, 2.2 eq.) was added slowly and the reaction mixture was stirred at room temperature for approximately 2 h after which time, analysis by TLC (10% EtOAc in hexanes) indicated essentially complete consumption of starting material. The mixture was partitioned between ethyl acetate (150 mL) and brine (150 mL). The organic phase was washed with saturated aqueous NaHCO₃ (2×150 mL), aqueous 10% citric acid (150 mL), and brine (150 mL). The organic layer was drained onto silica gel (˜10 g) and the mixture was concentrated to dryness under reduced pressure. Flash column chromatography (RediSepR_{f} SiO₂ (40 g), 100% hexanes→40% EtOAc in hexanes, monitoring at 220 and 254 nm and visualizing fractions with KMnO₄ stain) gave the OH-hexamer as a clear oil (3.7 g, 89%). FIG. 16

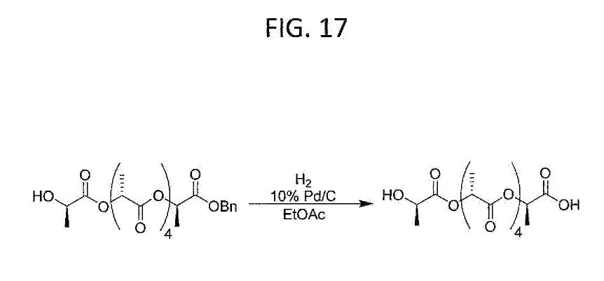

A suspension of the benzyl ester (3.4 g, 6.3 mmol) and 10% Pd/C (300 mg) in EtOAc (20 mL) was stirred overnight under a balloon of H₂ after which time analysis of the reaction mixture by TLC (30% EtOAc in hexanes) indicated complete consumption of starting material. The mixture was filtered through a pad of Celite. The pad was washed with EtOAc (2×10 mL) and the filtrate was concentrated to dryness under reduced pressure giving the hexamer as a clear pale oil that crystallized while on the high vacuum line overnight (2.6 g, 91%). FIG. 17

Testing D-Lactic Acid Oligomers to Sequester L-Lactate

Lactic acid concentration was measured by tetrazolium indicator color test strips (Accuvin, LLC, Napa, Calif.) from the reaction of L-lactic acid and nicotine-adenine in the presence of lactate dehydrogenase.

Samples of D-lactic acid oligomers (n=2, n=3, n=4, n=5, and n=6) were weighed on truncated 100 microliter pipette tips. The tips were then inserted into the pipette and D-lactic acid oligomers were mixed in 40 microliters of 10 mM L-lactic acid. The reactions were carried out at room temperature for 2 minutes, then 20 microliters were applied to L-lactic acid test strips, and the results were read after 2 minutes. (Tables 1 and 2) The molar ratio of D-lactic acid dimer to L-lactic acid was ˜0.004/0.004-0.006/0.004 for complete sequestration of L-lactic acid.

Stoichiometry of the L-lactic acid+D-lactic acid dimer stereocomplex reaction

TABLE 1
Stereocomplex reaction of L-lactic acid + D-lactic acid dimer

| Lactic acid | D-lactic acid dimer | Tetrazolium indicator |
|---|---|---|
| 40 microliters of 10 mM | 0 mg | greater than 400 mg/L |
| 40 microliters of 10 mM | 0.5 mg D-lactic acid dimer | 80 mg/L |
| 40 microliters of 10 mM | 1.0 mg D-lactic acid dimer | no color change |
| 40 microliters of 10 mM | 5.0 mg D-lactic acid dimer | no color change |
| 40 microliters of 10 mM | 7.0 mg D lactic acid dimer | no color change |

Reaction of L-lactate with n=3, n=4, n=5, and n=6 D-lactic acid oligomers

TABLE 2
Reaction of L-lactic acid with n = 3, n = 4, n = 5, and n = 6
D-lactic acid oligomers

| Lactic acid | D-lactic acid oligomer | Tetrazolium indicator |
|---|---|---|
| 40 microliters of 10 mM | 5.0 mg D-lactic acid n = 3 | greater than 400 mg/L |
| 40 microliters of 10 mM | 5.0 mg D-lactic acid n = 4 | greater than 400 mg/L |
| 40 microliters of 10 mM | 5.0 mg D-lactic acid n = 5 | greater than 400 mg/L |
| 40 microliters of 10 mM | 5.0 mg D-lactic acid n = 6 | greater than 400 mg/L |

### BENEFITS TO SOCIETY

In vitro, n=3, n=4, n=5, and n=6 D-lactic oligomers acid did not sequester L-lactate, however it is possible that one or more of these oligomers as a progdrug could be hydrolyzed to D-lactic acid dimer. If this occurs, these oligomers could be more potent medications, and these oligomers could be systemically administered.

### REFERENCES

Goldberg, J. S., Weinberg, J. B. (2014). U.S. Pat. No. 8,920,789 B2. Washington, D.C.: USPTO
Goldberg, J. S., Weinberg, J. B. (2016). U.S. Pat. No. 9,382,376 B2. Washington, D.C.: USPTO
Goldberg, J. S., Weinberg, J. B. (2017). U.S. patent application Ser. No. 18/0085,394 A1. Washington, D.C.: USPTO
Goldberg, J. S. (2015a) U.S. patent application Ser. No. 15/018,2481 A1. Washington, D.C.: USPTO
Goldberg J. S. PDLA a potential new potent topical analgesic: a case report. Local and regional anesthesia. 2014; 7:59-61.
Goldberg, J. S. (2015b) U.S. patent application Ser. No. 15/018,2553 A1. Washington, D.C.: USPTO
Perry J, Chambers A, Spithoff K, Laperriere N. Gliadel wafers in the treatment of malignant glioma: a systematic review. Current oncology. October 2007; 14(5):189-194.

Having described our invention, we claim:

1. A method to synthesize n=2, n=3, n=4, n=5, and n=6 D-lactic acid oligomers from methyl D-(+)-lactate.

2. The method of claim 1 where an equivalent, derivative, or analog of methyl D-(+)-lactate including D-lactic acid and D-lactate, are substituted for methyl D-(+)-lactate.

3. A method to treat cancer in a human subject by administering to said subject a prodrug of D-lactic acid dimer comprising an n=3, n=4, n=5, or n=6 D-lactic acid oligomer or a composite comprising n=3, n=4, n=5, and n=6 D-lactic oligomers.

4. The method of claim 3 to treat cancer and pain.

5. The method of claim 3 to treat pain.
